(19) United States
(12) Patent Application Publication
(10) Pub. No.: US 2004/0199129 A1
(43) Pub. Date: Oct. 7, 2004

(54) Vascular access port

(75) Inventor: Kristian DiMatteo, Watertown, MA (US)

Correspondence Address: PATRICK J. FAY, ESQ, FAY KAPLUN & MARCIN, LLP, 150 BROADWAY , SUITE 702, NEW YORK, NY, 10038, US

(73) Assignee: Scimed Life Systems, Inc., Maple Grove, MN (US)

(21) Appl. No.: 10/408,642

(22) Filed: Apr. 7, 2003

(51) Int. Cl. A61M 31/00

(52) U.S. Cl. 604/288.02

(57) ABSTRACT

The invention relates to a vascular access port for providing percutaneous access to the vasculature of a mammal. The vascular access port is formed from materials that preserve the structural integrity of the port and provide reduced degradation and distortion of a magnetic resonance image (MRI). The vascular access port includes at least one reservoir formed from MRI-compatible material(s) and a second material disposed within at least a portion of the at least one reservoir. Preferably, the second material is also MRI-compatible and more needle-impenetrable than the first material. Alternatively, the second material is substantially needle-impenetrable.

### TECHNICAL FIELD

The invention relates generally to devices for providing percutaneous access to the vasculature of a mammal, and more particularly to percutaneous access devices that provide reduced degradation and distortion of a magnetic resonance image (MRI).

### BACKGROUND INFORMATION

Various medical treatments require fluids, such as antibiotics, drugs, nutrition or chemotherapy agents, to be administrated directly into a patient's bloodstream. To facilitate such direct access to the bloodstream, a vascular access port is surgically implanted under the patient's skin and coupled to a central vein or artery. In general, vascular access ports include a reservoir which is accessed by inserting a needle through the patient's skin and penetrating a self-sealing septum on the top of the reservoir. Fluids introduced into the reservoir by the needle flow from the reservoir through a catheter and into a central vein or artery. Vascular access ports can also be used to withdraw fluids from a patient's body. Typically, conventional vascular access ports are surgically removed at the end of the treatment period or if damaged or malfunctioning.

In some cases, the needle, while penetrating the septum, unintentionally punctures the reservoir, resulting in fluid leakage and harm to the patient. To minimize this risk, many vascular access ports are made of a strong material, such as titanium. Use of titanium, however, distorts and degrades the quality of a magnetic resonance image (MRI) of the patient. Alternatively, other vascular access ports are made of polysulphone, which is lighter in weight and produces negligible, if any, MRI distortion. However, polysulphone access ports are less rigid, less durable, and weaker in strength than titanium ports, leaving them more susceptible to unintentional puncture by a needle. For this reason, there exists a need for an improved vascular access port.

### SUMMARY OF THE INVENTION

The invention relates to a vascular access port for providing percutaneous access to the vasculature of a mammal. In one embodiment, the vascular access port is formed from materials that preserve the structural integrity of the port and provide reduced degradation and distortion of a magnetic resonance image (MRI).

In one aspect of the invention, the vascular access port includes at least one reservoir/cavity formed from MRI-compatible material(s) and a second material disposed within at least a portion of the reservoir. Preferably, the second material is also MRI-compatible and more resistant to unintentional puncture, cracking or rupture by a needle inserted into the reservoir with typical force than the first material. In an alternate embodiment, the second material is substantially needle-impenetrable, meaning that it is strong and hard enough not to be penetrated, pierced or cracked by a needle inserted into the reservoir with typical force by, for example, a doctor, nurse or other person.

According to other embodiments, the first material is polysulphone. The second material can include at least one of ceramic, glass, graphite, silica, alumina, PTFE, nylon and copper. In one embodiment, the second material is in the form of a coating disposed on the first material within at least a portion of the reservoir.

In another embodiment, the reservoir(s) has a floor and an inner perimeter wall, which includes an upper portion and a lower portion, and the second material is disposed only on the floor and the lower portion of the inner perimeter wall. In a further embodiment, the second material forms a coating disposed on the floor and the lower portion of the inner perimeter wall. Alternatively, the second material can form an insert disposed within the reservoir.

In one embodiment, the insert forms a disc that interfits with the floor and the lower portion of the inner perimeter wall of the reservoir(s). In a further embodiment, the disc is formed from a second material that is substantially needle-impenetrable. In some embodiments, the disc is affixed to the reservoir.

According to another embodiment, the insert forms a cup that interfits with the floor and the upper and lower portions of the inner perimeter wall of the reservoir(s). In some embodiments, the cup is affixed to the reservoir. In a further embodiment, the cup is made from a material that is substantially needle-impenetrable.

Further embodiments of the invention include a vascular access port having a plurality of reservoirs and the plurality of reservoirs are integrally formed in a common base.

In another aspect, the invention provides a vascular access port including at least one reservoir, a septa for providing needle access to the reservoir, and a housing for covering the reservoir, while leaving the septa needle-accessible. The reservoir, septa and housing are formed from one or more MRI-compatible materials, and the MRI-compatible materials of the reservoir and the housing are substantially needle-impenetrable.

In one embodiment, the vascular access port includes a plurality of reservoirs and the plurality of reservoirs are affixed to a common base formed from one or more MRI-compatible materials. According to another embodiment, the plurality of reservoirs are integrally formed in a common base formed from one or more MRI-compatible materials.

### BRIEF DESCRIPTION OF THE DRAWINGS

An illustrative embodiment of the invention is explained in more detail with reference to the following drawings, which may not be drawn to scale.

FIG. 1 is a conceptual diagram depicting a dual reservoir vascular access port and catheter according to an illustrative embodiment of the invention and implanted in the chest of a human patient.

FIG. 2 is a conceptual diagram depicting a needle being inserted through a septum and into the reservoir of the illustrative vascular access port of FIG. 1.

FIG. 3 is a conceptual diagram depicting a medical professional observing the magnetic resonance imaging (MRI) of a patient having an implanted vascular access port of the type shown in FIG. 1.

FIG. 4 is an exploded view of the illustrated vascular access port of FIG. 1.

FIG. 5A is a top perspective view of the reservoirs of a vascular access port of the type depicted in FIG. 1, including a cup disposed within each reservoir according to an illustrative embodiment of the invention.

FIG. 5B is a top perspective view of the illustrative cup of FIG. 5A.

FIG. 6A is a top perspective view of the reservoirs of a vascular access port of the type depicted in FIG. 1, including a disc disposed within each reservoir according to an illustrative embodiment of the invention.

FIG. 6B is a top perspective view of the illustrative disc of FIG. 6A.

FIG. 7 is a top perspective view of the reservoirs of a vascular access port of the type depicted in FIG. 1, including a coating disposed within each reservoir according to an illustrative embodiment of the invention.

### ILLUSTRATIVE DESCRIPTION

This invention generally relates to a device for providing percutaneous access to the vasculature of a human or other mammalian body. In one embodiment, the invention provides an implantable vascular access port having a reservoir/cavity into which fluid may be introduced or withdrawn. According to one feature, the vascular access port includes one or more materials that reduce the likelihood of unintentional puncture, cracking, or rupture by a needle accessing the reservoir. According to another feature, these materials produce reduced degredation of the quality of magnetic resonance imaging (MRI).

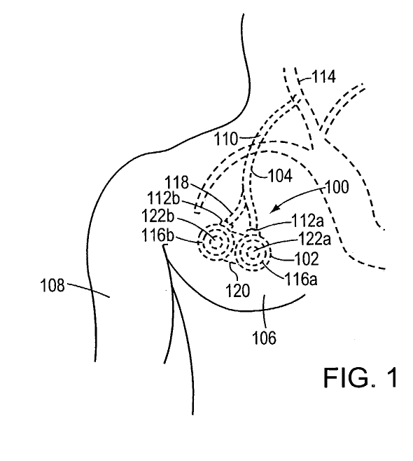

FIG. 1 depicts a system 100 including a vascular access port 102 coupled in fluid communication with a catheter 104 implanted within a chest region 106 of a human patient 108. As shown, a dual-lumen catheter tube 110 connects to the vascular access port 102 via catheter heads 112a and 112b to provide fluid communication between a central vein 114 and each reservoir 116a and 116b. In the illustrative embodiment of FIG. 1, the vascular access port 102 is shown having one reservoir 116a directly connected to the dual-lumen catheter tube 110 via the catheter head 112a, and another reservoir 116b connected to a side branch tube 118 of the dual-lumen catheter tube 110 via the catheter head 112b. A common base 120 is formed to define and shape each of the reservoirs 116a and 116b and to provide structural support for both reservoirs 116a and 116b. The reservoir 116a is accessed through a first septum 122a and the reservoir 116b is accessed through a second septum 122b to introduce or withdraw fluid from each reservoir respectively.

In alternate embodiments, the reservoirs 116a and 116b mechanically couple to the common base 120, which provides structural support for both reservoirs 116a and 116b. In other embodiments, the vascular access port 102 may include only a single reservoir or more than two reservoirs. For these alternate embodiments, the catheter tube generally has the same number of lumens as the number of reservoirs in the vascular access port. In further illustrative embodiments, the vascular access port includes more than one catheter tube, and the catheter tube may be permanently affixed to or detachable from the catheter heads.

The vascular access port 102 is typically implanted within a patient's chest through a surgical procedure performed in an operating room using general or local anesthesia. A first small incision is made to the patient's skin in his or her chest region and the vascular access port is placed underneath his or her skin and the underlying muscle. A second incision is made to the patient's skin near his or her collarbone and a third incision is made to a vein or artery located in the lower part of the patient's neck, such as the superior vena cava. The catheter's distal end is placed into the vein or artery via the second and third incisions and the catheter's proximal end is tunneled under the patient's skin towards the first incision, where it is connected to the vascular access port via the catheter heads. In one embodiment, the catheter's distal end is passed through the patient's vein or artery and into an atrium of the patient's heart. The proper function of the vascular access port can be tested by injecting fluid into the reservoirs of the vascular access port, and if the vascular access port is working properly, both the first and second skin incisions are closed, typically leaving two small scars on the patient's skin.

In an alternative exemplary implantation procedure, the catheter's proximal end is connected to the vascular access port before the vascular access port is implanted in the patient's chest region. One incision is made to the patient's chest region and the vascular access port and catheter are placed underneath the skin and underlying muscle. The catheter's distal end is tunneled under the patient's skin towards a vein or artery located near the patient's lower neck, and the vein or artery is incised percutaneously. The catheter's distal end is inserted into the vein or artery and can be passed into an atrium of the patient's heart.

Additionally, although the illustrative embodiment depicts the system 100 as being implanted in the chest region 106, the system 100 is capable of being implanted in other locations throughout the body, for example, in a patient's arm, forearm and/or upper back, depending on the size and intended use of the vascular access port. Furthermore, the vascular access port 102 may have a loop or other exterior element that allows the vascular access port to be sutured to an underlying muscle.

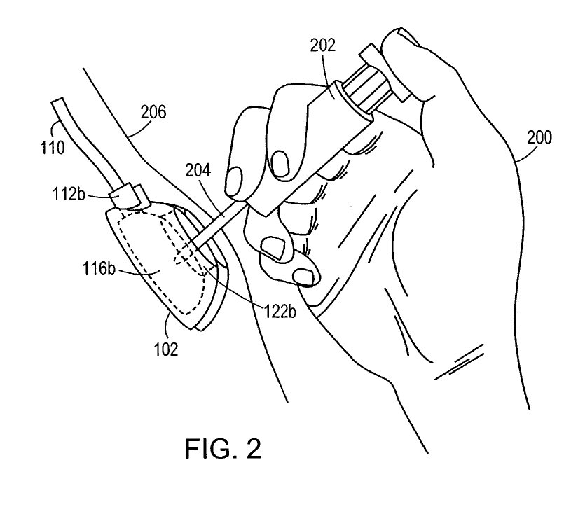

FIG. 2 depicts a hand 200 holding a syringe 202 in fluid communication with a needle 204 which pierces a patient's skin 206 and penetrates the septum 122b to deliver or withdraw fluids from the reservoir 116b. Any fluid delivered to the reservoir 116b flows through the dual-lumen catheter tube 110 via the catheter head 112b and into the central vein 114 of the human patient 108. The needle's 204 point can be designed to prevent damage to the septum 122b when penetrating the septum 122b and accessing the reservoir 116b, and the needle may be, for example, a Huber needle.

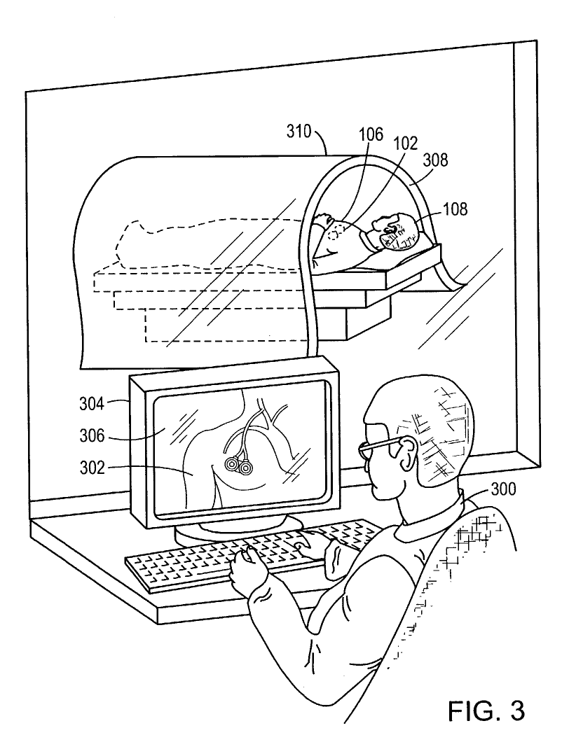

FIG. 3 depicts a medical professional 300 observing an image 302 of a human patient 108 having a vascular access port 102 implanted in his or her chest region 106. The image 302 is displayed via a computer 304 and an associated display device 306. In the illustrative embodiment, the human patient 108 lays down horizontally on his or her back inside a closed tunnel 308 of an MRI machine 310. The MRI machine 310, and generally any MRI machine, uses a magnet, radio waves, a computer 304 and an associated display device 306 to collect and process images.

In alternate embodiments, MRI machines with less confining tunnels or cylinders are used. Any type of MRI machine enables the medical professional 300 to view and diagnose conditions of the patient's anatomy surrounding the vascular access port 102, such as cardiovascular disease, cancer or a bone disorder, as well as conditions of the vascular access port itself, such as occlusion, rupture, puncture, detachment or infection.

MRI machines apply a magnetic field to create a visible image. When the magnetic field is applied, different materials typically react with different magnetic forces and torques, depending on each material's magnetic susceptibility. The reaction produces radio waves that can be interpreted by a computer. Depending on the strength of these radio waves, the computer assesses different positions of each material within the magnetic field, and uses scales of color to create an MRI image. Materials with strong magnetic susceptibility can cause the computer to detect erroneous positions and produce abnormal contrasting values. Thus, as the strength of the material's magnetic susceptibility increases, the image quality degrades and the image becomes more distorted. In certain circumstances, a medical professional's ability to assess the condition of the patient's anatomy surrounding a vascular access port is impeded by the poor quality, distortion and degradation of the MRI image. Furthermore, a medical professional's ability to assess the condition of the vascular access port can also be impeded by the poor quality, distortion and degradation of the MRI image.

Magnetic susceptibility is a measure of the degree to which a material can perturb a magnetic field. Maximum perturbation, which is a dimensionless unit, is expressed by the formula: X=ΔBₘₐₓ/B₀, wherein B is the magnetic induction or magnetic flux density (units are tesla), often referred to as the magnetic field; B₀ is the static magnetic field in the MRI machine; and ΔB is the perturbation in B produced by a magnetized object.

Materials are considered not to be MRI-compatible if they have a magnetic susceptibility greater than about 178×10⁻⁶. Such materials cause substantial degradation and distortion in MRI images, and include, for example, titanium, stainless steel, platinum, and chromium. These non MRI-compatible materials may also become strongly magnetized by the magnetic field of an MRI machine, and a vascular access port made of these materials may cause a magnetic quench and/or damage to the patient if the vascular access port is pulled toward the magnet of an MRI machine when the magnetic field is applied.

Other materials, such as polysulphone, have weak magnetic susceptibility and do not exhibit strong forces and torques when subjected to or placed near the magnetic field of an MRI machine, resulting in less distortion and degradation of the MRI image. In general, materials that produce limited or negligible MRI image distortion or degradation have a magnetic susceptibility less than about 178×10⁻⁶, and materials within this range are considered MRI-compatible. For example, materials included within the ceramic group, such as silica, alumina, and silicon nitride, have a magnetic susceptibility of about −9×10⁻⁶ to about −20×10⁻⁶, which puts them in the class of materials that are MRI-compatible. Other MRI-compatible materials include, for example, nylon, PTFE (teflon®), zirconia, poly-ether-ether-ketone, glass, wood and copper. Human tissue also produces limited or negligible MRI image distortion, having a magnetic susceptibility in the range of about −7×10⁻⁶ to about −11×10⁻⁶.

Table 1 contains an exemplary, non-exclusive list of some additional MRI-compatible materials that exhibit insignificant forces and torques when near the magnetic field of an MRI machine.

TABLE 1

| Material | Susceptibility (×10⁻⁶) |
|---|---|
| Phosphorus (red) | −18.5 |
| Alumina | −18.1 |
| Silica | −16.3 |
| Lead | −15.8 |
| Zinc | −15.7 |
| Pyrex Glass (Corning 7730) | −13.88 |
| Sulfur (α) | −12.6 |
| Sulfur (β) | −11.4 |
| Magnesia | −11.4 |
| Copper | −9.63 |
| Water (37 degrees) | −9.05 |
| Human Tissues | ˜(−11.0 to −7.0) |
| Silicon Nitride | ˜−9.0 |
| Graphite (parallel to atomic planes) | −8.5 |
| Zirconia | −8.3 |
| Whole Blood (deoxygenated) | −7.90 |
| Germanium | −6.52 |
| Red blood cell (deoxygenated) | −6.52 |
| Silicon | −4.2 |
| Liver (severe iron overload) | ˜0.0 |
| Hemoglobin Molecule (deoxygenated) | +0.15 |
| Air (NTP) | 0.36 |
| Tin (β-white) | 2.4 |
| Rubidium | 3.8 |
| Cesium | 5.2 |
| Potassium | 5.8 |
| Sodium | 8.5 |
| Magnesium | 11.7 |
| Yttria | 12.4 |
| Aluminum | 20.7 |
| Calcium | 21.7 |
| Tungsten | 77.2 |
| Zirconium | 109 |
| Nickel chloride in water | 116 |
| Yttrium | 119 |
| Molybdenum | 123 |
| Rhodium | 169 |
| Tantalum | 178 |

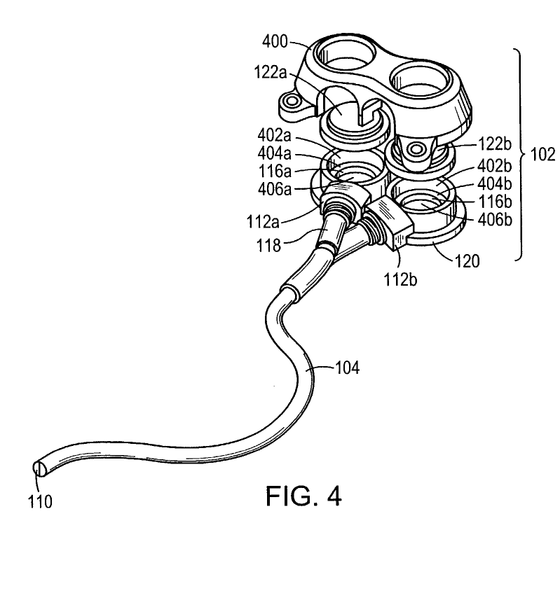

FIG. 4 depicts an exploded view of the vascular access port 102 of the type in FIG. 1. As shown in FIG. 4, the vascular access port 102 includes a common base 120, reservoirs 116a and 116b, septa 122a and 122b, and a cover/housing 400. The first septum 122a sits on top and effectively seals the first reservoir 116a and the second septum 122b sits on top and effectively seals the second reservoir 116b. The cover/housing 400 is shaped to allow a needle 204 to access the top of each septum 122a and 122b, while covering the base 120, the outside perimeters of the reservoirs 116a and 116b, the outside perimeters of the septa 122a and 122b, and the catheter heads 112a and 112b. The dual-lumen catheter tube 110 is in fluid communication with the reservoirs 116a and 116b via the catheter heads 112a and 112b.

According to one embodiment, the base 120 is formed out of a polymer, such as polysulphone, using a molding press to define and shape the reservoirs 116a and 116b within the base 120. The base 120 may also be formed by injection molding, or can be cast, milled or assembled. Alternatively, the base 120 may be formed by any process suitable for forming plastics or ceramics. In another embodiment, the base 120 is formed out of a material, such as ceramic, which provides a lower risk of unintentional puncture, cracking or rupture by a needle inserted into the reservoirs 116a and 116b with typical force than polysulphone does, and is MRI-compatible. In a further embodiment, the base 120 is formed out of a material that is substantially needle-impenetrable, in that it is strong and hard enough not to puncture, crack or rupture when contacted by a needle inserted with typical force by, for example, a doctor, nurse, or other person, and is MRI-compatible.

According to the illustrative embodiment of FIG. 4, the reservoirs 116a and 116b have identical circular inner perimeter walls 402 and 404, which include an upper portion 402 and a lower portion 404, and a floor 406. The inner perimeter walls 402 and 404 allow the reservoirs 116a and 116b to hold fluid. In alternate embodiments, the reservoirs 116a and 116b are not identical, or have different shapes and sizes depending on the medical treatment necessary. In a further embodiment, the reservoirs 116a and 116b are designed to eliminate dead space, corners or residue, such as thrombus or sludge, that can predispose the vascular access port and/or patient to infection.

The septa 122a and 122b are partitions or membranes that close the reservoirs 116a and 116b and are made out of a dense, MRI-compatible, self-sealing material, such as silicone, that is capable of gripping a needle 204 and withstanding repeated access for several years. The top of each septum 122a and 122b is palpable through the patient's skin 206, and can have a rounded surface or a protruding rim. In the illustrative embodiment, the septa 122a and 122b are separated into individual components. In alternate embodiments, the septa are connected to form a one-piece unit.

As shown in FIG. 4, the cover/housing 400 is formed as a one-piece unit. Alternatively, the cover/housing can be made as two or more individual components, wherein each cover/housing individually covers a septum and a reservoir, and the number of cover/housing components depends on the number of septa and reservoirs in the vascular access port. In one embodiment, the cover/housing 400 is formed out of an MRI compatible material, such as polysulphone. In another embodiment, the cover/housing 400 is formed out of a material that provides a lower risk of unintentional puncture, cracking or rupture by a needle inserted with typical force than polysulphone, and is MRI-compatible. In a further embodiment, the cover/housing 400 is formed out of a material that is substantially needle-impenetrable, and is MRI-compatible.

In the illustrative embodiment of FIG. 4, the catheter 104 includes a side branch tube 118 and a dual-lumen catheter tube 110, which allows fluid placed in one reservoir 116a to be delivered to the central vein 114 separately from fluid placed in the other reservoir 116b. The catheter 104 extends from the catheter heads 112a and 112b in varying lengths according to the length needed to reach its destination, and can be made of pliable materials, such as silastic or polyurethane. In one embodiment, the catheter heads 112a and 112b are made of an MRI-compatible material, such as polysulphone. In another embodiment, the catheter heads 112a and 112b are made of a material that provides a lower risk of unintentional puncture, cracking or rupture by a needle inserted with typical force than polysulphone, and is MRI-compatible. In a further embodiment, the catheter heads 112a and 112b are formed out of a material that is substantially needle-impenetrable, and is MRI-compatible. In alternate embodiments, the catheter tube may include only one lumen, more than two lumens, or have a valve-tip or an end-hole, depending on the number of reservoirs needed or the treatment prescribed. In further embodiments, more than one catheter tube is connected to the vascular access port, and the catheter tube may be permanently affixed to or detachable from the catheter heads.

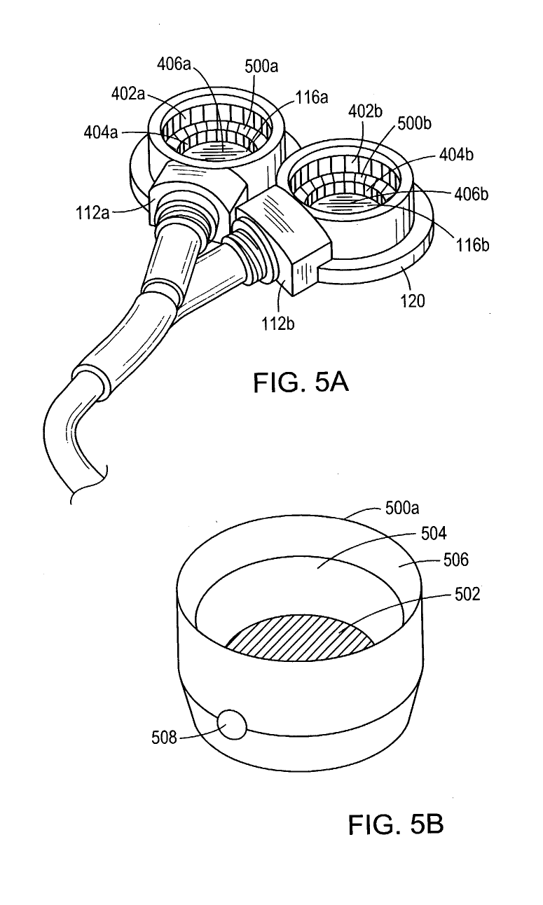

FIG. 5A is a top perspective view of the reservoirs 116a and 116b of a vascular access port of the type depicted in FIG. 1. In the illustrative embodiment, a first cup 500a and a second cup 500b are disposed within the reservoirs 116a and 116b, respectively. The walls of the cups 500a and 500b conform to the shape of the reservoirs 116a and 116b, such that the cups 500a and 500b match/interfit with the inner perimeter walls 402 and 404 and the floor 406 of the reservoirs 116a and 116b, and provide for fluid communication between the catheter heads 112a and 112b and the reservoirs 116a and 116b, respectively, when placed inside the reservoirs 116a and 116b. The walls of the cups 500a and 500b extend to the top end of the upper portions 402a and 402b of the inner perimeter walls 402 and 404. The septa 122a and 122b fit on top of each cup 500a and 500b, creating a tight seal sufficient to prevent fluid from leaking out of the reservoirs 116a and 116b.

In an alternate embodiment, the walls of the cups 500a and 500b extend to the upper portions 402a and 402b of the inner perimeter walls 402 and 404, and the septa 122a and 122b fit on top of each reservoir 116a and 116b, creating a tight seal against the top end of the upper portions 402a and 402b of the inner perimeter walls 402 and 404 sufficient to prevent fluid from leaking out of the reservoirs 116a and 116b.

In one embodiment, the common base 120, reservoirs 116a and 116b, catheter heads 112a and 112b, and cover/housing 400 are made of a first material that is MRI-compatible, such as polysulphone. The cups 500a and 500b are made of a second material that is less susceptible to unintentional puncture, cracking or rupture by a needle inserted into the reservoir with typical force than the first material, and is MRI-compatible, such as ceramic, graphite, silica, alumina, PTFE (teflon®), nylon or copper. Alternatively, the second material can comprise a high-impact glass, such as PYREX® glass, manufactured by Corning Incorporated. Preferably, the second material is substantially needle-impenetrable, in that it is strong and hard enough not to puncture, crack or rupture when contacted by a needle inserted with typical force by, for example, a doctor, nurse or other person, and is MRI-compatible. In a further illustrative embodiment, the cups 500a and 500b can withstand single or continuous forceful impact by a needle. In a preferred embodiment, the second material is compatible with chemotherapy agents.

The cups 500a and 500b can be, for example, molded, cast, or fluid-injected into the reservoirs 116a and 116b, respectively. Alternatively, the cups 500a and 500b are bonded, adhered, glued or affixed in some like manner into the reservoirs 116a and 116b, respectively. In another embodiment, the cups 500a and 500b are milled or assembled and mechanically affixed into the reservoirs 116a and 116b.

In an alternate embodiment, the reservoirs 116a and 116b do not include cups 500a and 500b, and the common base 120, reservoirs 116a and 116b, catheter heads 112a and 112b, and cover/housing 400 are made of a material that is less susceptible to unintentional puncture, cracking or rupture by a needle inserted with typical force than polysulphone and produces less MRI image distortion or degradation than titanium.

FIG. 5B depicts an illustrative example of the first cup 500a of FIG. 5A. As shown, the cup 500a is shaped to include a floor 502, a first cylindrical wall segment 504, and a second cylindrical wall segment 506, such that the floor 502 of the cup 500a matches/interfits with the floor 406a of the reservoir 116a and the cylindrical wall segments 504 and 506 match/interfit with the inner perimeter walls 402a and 404a of the reservoir 116a. In the illustrative embodiment, an aperture 508 provides for fluid communication between the catheter heads 112a and 112b and the cup 500a. The first cylindrical wall segment 504 extends from the floor 502 of the cup to the lower end of the second cylindrical wall segment 506, and the second cylindrical wall segment 506 extends from the top end of the first cylindrical wall segment 504 to the top end of the upper portion 402a of the inner perimeter wall 402a and 404a of the reservoir 116a, allowing the cup to hold fluid.

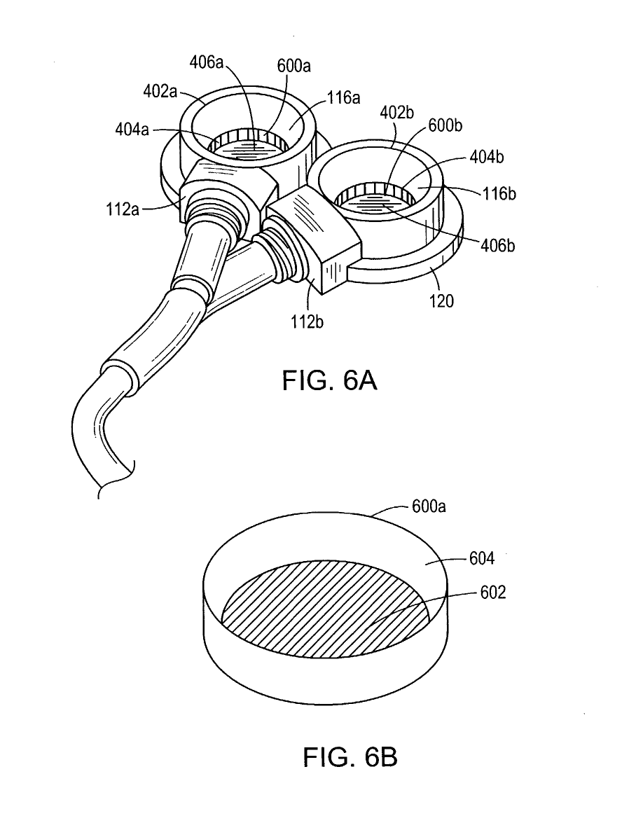

FIG. 6A is a top perspective view of the reservoirs 116a and 116b of a vascular access port of the type depicted in FIG. 1. In the illustrative embodiment, a first disc 600a is disposed within the reservoir 116a and a second disc 600b is disposed within the reservoir 116b. The walls of the discs 600a and 600b conform to the shape of the reservoirs 116a and 116b, such that the discs 600a and 600b match/interfit with the lower portion 404 of the inner perimeter walls 402 and 404 and floor 406 of the reservoirs 116a and 116b when placed inside the reservoirs 116a and 116b. The septa 122a and 122b fit on top of each reservoir 116a and 116b, creating a tight seal sufficient to prevent leakage from the reservoirs 116a and 116b.

In one embodiment, the common base 120, reservoirs 116a and 116b, catheter heads 112a and 112b, and cover/housing 400 are made out of a first material that is MRI-compatible, such as polysulphone. The discs 600a and 600b are made out of a second material that is less susceptible to unintentional puncture, cracking or rupture by a needle inserted with typical force than the first material, and is MRI-compatible, such as ceramic, graphite, silica, alumina, PTFE (teflon®), nylon or copper. Alternatively, the second material can comprise a high-impact glass, such as PYREX® glass. In another embodiment, the second material is substantially needle-impenetrable, in that it is strong and hard enough not to puncture, crack or rupture when contacted by a needle inserted with typical force by, for example, a doctor, nurse or other person, and is MRI-compatible. Preferably, the second material is compatible with chemotherapy agents. In a further embodiment, the discs 600a and 600b withstand single or continuous forceful impact by a needle.

The discs 600a and 600b can be, for example, molded, cast, or fluid-injected into the reservoirs 116a and 116b, respectively. Alternatively, the discs 600a and 600b are bonded, adhered, glued or affixed in some like manner into the reservoirs 116a and 116b, respectively. In another embodiment, the discs 600a and 600b are milled or assembled and mechanically affixed into the reservoirs 116a and 116b.

FIG. 6B depicts an illustrative example of the first disc 600a of FIG. 6A. As shown, the disc 600a is shaped to include a floor 602 and a cylindrical wall unit 604, such that the floor 602 of the disc 600a matches/interfits with the floor 406a of the reservoir 116a, and the cylindrical wall unit 604 matches/interfits with the lower portion 404a of the inner perimeter walls 402 and 404 of the reservoir 116a.

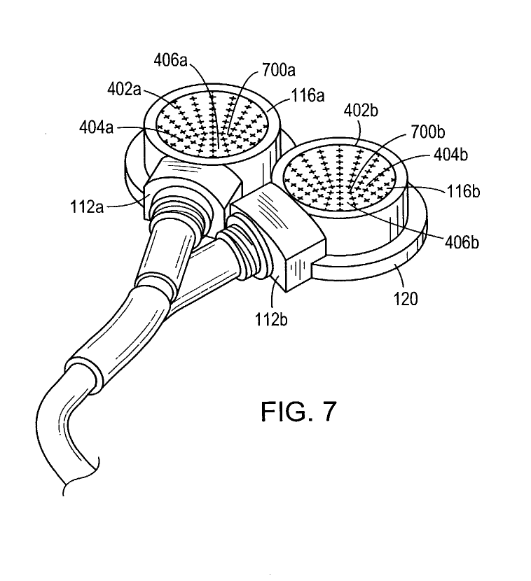

FIG. 7 is a top perspective view of the reservoirs 116a and 116b of a vascular access port of the type depicted in FIG. 1. In the illustrative embodiment, a first coating 700a is disposed within the reservoir 116a and a second coating 700b is disposed within the reservoir 116b. The coating 700a and 700b covers the floor 406 and extends to the upper portion 402 of the inner perimeter walls 402 and 404 of the reservoirs 116a and 116b, and provides for fluid communication between the catheter heads 112a and 112b and the reservoirs 116a and 116b, respectively. The septa 122a and 122b fit on top of each reservoir 116a and 116b, creating a tight seal against the upper portion 402 of the inner perimeter walls 402 and 404 sufficient to prevent fluid from leaking out of the reservoirs 116a and 116b. In an alternate embodiment, the coating 700a and 700b extends to the top end of the upper portion 402 of the inner perimeter walls 402 and 404, and the septa 122a and 122b sit on top of each reservoir 116a and 116b, creating a tight seal against the coating 700a and 700b sufficient to prevent fluid from leaking out of the reservoirs 116a and 116b.

In one embodiment, the common base 120, reservoirs 116a and 116b, catheter heads 112a and 112b, and cover/housing 400 in FIG. 7 are made out of a first material that is MRI-compatible, such as polysulphone. The coating 700a and 700b is made out of a second material that is less susceptible to unintentional puncture, cracking or rupture by a needle inserted with typical force than the first material and is MRI-compatible, such as ceramic, graphite, silica, PTFE (teflon®), nylon, copper, a polymer composite, or a high-impact glass, such as PYREX® glass. Preferably, the second material is substantially needle-impenetrable, in that it is strong and hard enough not to puncture, crack or rupture when contacted by a needle inserted with typical force by, for example, a doctor, nurse or other person, and is MRI-compatible. In a further illustrative embodiment, the coating 700a and 700b can withstand single or continuous forceful impact by a needle. In a preferred embodiment, the coating 700a and 700b is compatible with chemotherapy agents. The coating 700a and 700b can be, for example, molded, cast, or fluid-injected into the reservoirs 116a and 116b, respectively. Alternatively, the coating 700a and 700b can be, for example, sprayed, adhered, or glued into the reservoirs 116a and 116b, respectively.

In an alternate embodiment, the common base 120, reservoirs 116a and 116b, catheter heads 112a and 112b, cover/housing 400 and septa 122a and 122b are made out of the same or various different MRI-compatible materials.

Variations, modifications, and other implementations of what is described herein will occur to those of ordinary skill in the art without departing from the spirit and the scope of the invention as claimed. Accordingly, the invention is to be defined not by the preceding illustrative description but instead by the spirit and scope of the following claims and all equivalents thereof.

What is claimed is:

1. A vascular access port comprising:
at least one reservoir formed from a first material, the first material being MRI-compatible; and
a second material disposed within at least a portion of the at least one reservoir, the second material being MRI-compatible and more resistant to penetration by a needle than the first material.

2. The vascular access port of claim 1, wherein the second material is substantially needle-impenetrable.

3. The vascular access port of claim 1, wherein the first material is polysulphone.

4. The vascular access port of claim 1, wherein the second material includes at least one of ceramic, glass, graphite, silica, alumina, PTFE, nylon and copper.

5. The vascular access port of claim 1, wherein the second material is a coating disposed on the first material within at least a portion of the at least one reservoir.

6. The vascular access port of claim 1, wherein the at least one reservoir has a floor and an inner perimeter wall, which includes an upper portion and a lower portion, and the second material is disposed only on the floor and the lower portion of the inner perimeter wall.

7. The vascular access port of claim 6, wherein the second material forms a coating disposed on the floor and the lower portion of the inner perimeter wall.

8. The vascular access port of claim 6, wherein the second material forms an insert disposed within the reservoir.

9. The vascular access port of claim 8, wherein the insert is a disc formed to interfit with the floor and the lower portion of the inner perimeter wall.

10. The vascular access port of claim 9, wherein the disc is affixed to the reservoir.

11. The vascular access port of claim 9, wherein the second material is substantially needle-impenetrable.

12. The vascular access port of claim 8, wherein the insert includes a cup formed to interfit with the floor and the upper and lower portions of the inner perimeter wall.

13. The vascular access port of claim 12, wherein the cup is affixed to the reservoir.

14. The vascular access port of claim 12, wherein the second material is substantially needle-impenetrable.

15. The vascular access port of claim 1, wherein the at least one reservoir is a plurality of reservoirs and the plurality of reservoirs are integrally formed in a common base.

16. A vascular access port comprising:
at least one reservoir;
a septa for providing needle access to the at least one reservoir; and
a housing for covering the at least one reservoir while leaving the septa needle accessible;
wherein the at least one reservoir, septa and housing are formed from one or more MRI-compatible materials, and the MRI-compatible materials of the reservoir and the housing are also substantially needle-impenetrable materials.

17. The vascular access port of claim 16, wherein the at least one reservoir is a plurality of reservoirs and the plurality of reservoirs are affixed to a common base formed from one or more MRI-compatible materials.

18. The vascular access port of claim 16, wherein the at least one reservoir is a plurality of reservoirs and the plurality of reservoirs are integrally formed in a common base formed from one or more MRI-compatible materials.
